(19) United States
(12) Patent Application Publication
(10) Pub. No.: US 2024/0403716 A1
(43) Pub. Date: Dec. 5, 2024

(54) METHOD AND APPARATUS FOR IDENTIFYING A CHEMICAL COMPOUND

(71) Applicant: ANOR TECHNOLOGIES PTE. LTD., Singapore (SG)

(72) Inventors: Nan ZHANG, Singapore (SG); Yee Fun LIM, Singapore (SG); Zi Xi Josie LIM, Singapore (SG); Wei Ji PHUA, Singapore (SG)

(21) Appl. No.: 18/697,736

(22) PCT Filed: Jun. 20, 2022
(86) PCT No.: PCT/SG2022/050425

§ 371 Date: Apr. 2, 2024

(30) Foreign Application Priority Data
Jun. 21, 2021 (WO) PCT/SG2021/050359

(51) Int. Cl. G06N 20/00; G06N 7/01

(52) U.S. Cl. CPC G06N 20/00; G06N 7/01

(57) ABSTRACT

The apparatus (100A) comprises a cover plate (102) for focusing a terahertz beam (108) and a bottom plate (104) for enhancing an absorption signal of a sample (106) to the terahertz beam (108). The apparatus (100A) comprises a terahertz time-domain spectroscopy module (110) for acquiring (802) terahertz time-domain spectroscopy (THz TDS) data. The apparatus (100A) comprises a processor (120) configured to implement a trained machine learning model (130) that is trained on training dataset (114) comprising a pure compound dataset (114a) and a mixture dataset (114b). The trained machine learning model (130) is configured to process (804) an acquired THz TDS data (116) for obtaining a reduced dimensionality dataset (118); extract (806) at least one feature (118a) from the reduced dimensionality dataset (118); generate (808) a classification label (122) for the reduced dimensionality dataset (118); and indicate (810) a probability of identifying the chemical compound (112).

The present application claims earlier the filing date of an earlier patent application Nr. PCT/SG2021/050359 as its priority date, which was filed on 21 Jun. 2021. Content or subject matter of the priority application is hereby incorporated entirely or wherever appropriate.

An example of the present invention generally relates to a method and apparatus for identifying a chemical compound.

Over the past few years many different techniques are being used to identify different types of chemical substances. The detection of the chemical substances is performed at different places and the detection techniques and instrument may vary according to situational attributes. For example, there may be a situation of detecting chemical substances in military and civilian defense resources. Different types of devices or portable instruments may be carried to perform the detection in such places to understand the severity and extent of a hazard that may be related to the chemical substance. Another example application may include screening for illicit drugs at customs checkpoints. Although state-of-the-art instrumentation exist for characterizing chemical compounds (such as Nuclear Magnetic Resonance and Infra-Red techniques), these are mostly based on large equipment that are housed in the laboratory and are non-portable and cannot be deployed on-site. Some portable infrared instruments are available commercially, for instance those based on mid-infrared (MIR) Fourier transform or Raman spectroscopic techniques. However, infrared (IR) excitation can induce a phase change, or initiate photochemical reactions, in samples being investigated, and fluorescence from the sample may swamp any IR signal. Further, packaging material could absorb the infrared signal and prevent it from reaching the sample within. Thus, more reliable identification techniques are needed.

Terahertz time-domain spectroscopy (THz-TDS) may also be used to perform a sample detection method. However, the identification and analysis of the THz spectra is fairly complicated with multiple peaks of varying amplitudes, and different compound spectra can have overlapping peaks in similar wavelength range. Making identification even more challenging is the fact that compounds can often exist in mixtures and not in pure states.

Therefore, it is desirable to distinguish between different compounds with an automatic detection method on equipment that may be operated by non-expert users.

The present invention aims to provide new and useful method and apparatus for identifying a chemical compound. Essential features are provided by one or more independent claims, whilst advantageous features are presented by their dependent claims.

In order to solve the foregoing problem, an aspect of the present invention provides a method for identifying a chemical compound from a sample. The method includes acquiring terahertz time-domain spectroscopy (THz TDS) data of a sample. The method further includes processing the acquired THz TDS data for obtaining a reduced dimensionality dataset associated with the THz TDS data by using a trained machine learning model. The trained machine learning model is trained on a training dataset comprising at least one pure compound dataset and at least one mixture dataset. The method further includes extracting at least one feature from the reduced dimensionality dataset by using the trained machine learning model, the at least one feature comprising at least an absorption spectra parameter. The method further includes generating a classification label for the reduced dimensionality dataset based on the extracted at least one feature using the trained machine learning model and indicating (e.g., displaying, showing, telling, pronouncing, expressing, etc.), by the trained machine learning model, at least one name of the identified chemical compound according to identification probability of the chemical compound in the sample, based on the classification label generated.

To that end, the trained machine learning model may perform logical operations by involving supervised machine learning, unsupervised as well as automated machine learning. The trained machine learning model is configured to generate the classification label for the reduced dimensionality dataset based on the extraction at least one feature.

According to an example, the method includes determining at least one of a presence of the chemical compound in the sample and an absence of the chemical compound in the sample, based on the indication of the probability of identification of the chemical compound.

According to an example, the absorption spectra parameter depends on thickness “d” of the sample.

To that end, absorption spectra correspond to a spectrum that is constituted by frequencies of light transmitted with dark bands when electrons absorb energy in the ground state to reach higher energy states. This type of spectrum is produced when atoms absorb energy.

According to an example, processing the acquired THz TDS data for obtaining the reduced dimensionality dataset by using the trained machine learning model further comprises: transforming, using a dimensionality reduction algorithm, the acquired THz TDS data from a high dimension to a low dimension in a frequency domain, the low dimension being lesser than the high dimension. The dimensionality reduction algorithm comprises at least one of a principal component analysis algorithm or a t-distributed stochastic neighbour embedding algorithm.

To that end, the high dimension in the frequency domain of the acquired THz TDS data 116 is 100 Gigahertz (GHz). In addition, the low dimensional in the frequency domain of the acquired THz TDS data 116 is 10 Terahertz (THz). Further, the high dimension and the low dimension may not be limited to the above mentioned range. The reduced dimensionality dataset is obtained by filtering multiple THz spectrums from the acquired THz TDS data based on predetermined frequency bands. The multiple THz spectrums are filtered when the same frequency bands are processed with uniform resolution by cubic spline interpolation. In an example, the acquired THz TDS data may also be filtered to remove the noise, where a smoothing filter may be applied to remove the noise without distorting the overall spectra

According to an example, the generation of the classification label for the reduced dimensionality dataset, by the trained machine learning model, further comprises: processing the reduced dimensionality dataset by a machine learning classification algorithm associated with the trained machine learning model. The machine learning classification algorithm comprising at least one of: a support vector classifier algorithm, a random forest algorithm, a gradient boosting classifier algorithm, and a neural network classifier algorithm.

According to an example, the pure compound dataset comprises data associated with the THz TDS data for the chemical compound when the chemical compound exists in a pellet form.

To that end, the pure compound dataset is comprised of a particular set of molecules or ions that are held together by chemical bonds which cannot be broken. In addition, these particular set of molecules or ions cannot be decomposed by ordinary chemical methods, such as the application of heat.

According to an example, the mixture dataset comprises data associated with the THz TDS data for the chemical compound when the chemical compound exists in a powder form. To that end, the mixture dataset is comprised of two or more different substances, not chemically joined together.

According to an example, the classification label generated for the reduced dimensionality dataset comprises at least one of a true label and a false label. The true label is indicative of the presence of the chemical compound in the sample, and the false label is indicative of the absence of the chemical compound in the sample.

According to an example, the method further includes calculating predictive probabilities and confusion matrices for a support vector classifier algorithm, a random forest algorithm, a gradient boosting classifier algorithm, and a neural network classifier algorithm for training the trained machine learning model.

According to an example, the method further comprises obtaining confidence level of the predictive probabilities by implementing a neural network classifier algorithm having a categorical cross-entropy function, wherein the categorical cross-entropy function provides a measure of the confidence level of the predictive probabilities.

To that end, the trained machine learning model is implemented with the neural network classifier algorithm having a categorical cross-entropy function to obtain the confidence level of the predictive probabilities. The categorical cross-entropy function provides a measure of the confidence level of the predictive probabilities.

According to an example, the method further comprises: transforming the acquired THz TDS data from a time domain to a frequency domain; and processing the THz TDS data that is transformed. The THz TDS data that is transformed is processed for obtaining the reduced dimensionality dataset.

To that end, the terahertz time-domain spectroscopy (THz TDS) is performed based upon attenuated total reflection. The attenuated total reflection is a contact sampling technique that is used mostly in Fourier Transform-IR Spectroscopy to provide a quick, non-destructive sampling.

According to an example, the classification label generated for the reduced dimensionality dataset comprises at least one of a true label and a false label, wherein the true label is indicative of the presence of the chemical compound in the sample, and the false label is indicative of the absence of the chemical compound in the sample.

To that end, the true label and false label provides accurate indication of presence or absence of the chemical compound in the sample.

Another aspect of the present invention provides an apparatus for identifying a chemical compound from a sample. The apparatus comprises a cover plate for focusing a terahertz beam. The apparatus further comprises a bottom plate for enhancing an absorption signal of the sample to the terahertz beam. The sample is configured to be sandwiched between the cover plate and the bottom plate for absorbing the terahertz beam. Furthermore, the apparatus comprises a terahertz time-domain spectroscopy module for acquiring terahertz time-domain spectroscopy (THz TDS) data for the sample. The apparatus further comprises a processor configured to implement a trained machine learning model. The trained machine learning model being trained on a training dataset comprising at least one pure compound dataset and a mixture dataset. The trained machine learning model is configured to process the acquired THz TDS data for obtaining a reduced dimensionality dataset associated with the THz TDS data; extract at least one feature from the reduced dimensionality dataset. The at least one feature comprise at least an absorption spectra parameter; generate a classification label for the reduced dimensionality dataset based on the extraction of at least one feature; and indicate a probability of identifying the chemical compound in the sample, based on the classification label generated.

According to an example, the processor in combination with the trained machine learning model is further configured to determine at least one of a presence of the chemical compound in the sample and an absence of the chemical compound in the sample, based on the indication of the probability of identifying the chemical compound in the sample.

According to an example, the pure compound dataset comprises data associated with the THz TDS data for the chemical compound when the chemical compound exists in a pellet form.

According to an example, the mixture dataset comprises data associated with the THz TDS data for the chemical compound when the chemical compound exists in a powder form.

According to an example, the classification label generated for the reduced dimensionality dataset comprises at least one of a true label and a false label, wherein the true label is indicative of the presence of the chemical compound in the sample, and the false label is indicative of the absence of the chemical compound in the sample.

The foregoing summary is illustrative only and is not intended to be in any way limiting. In addition to the illustrative aspects, examples, and features described above, further aspects, examples, and features will become apparent by reference to the drawings and the following detailed description.

In various aspects, a method, and an apparatus for identifying chemical compound is provided. The method is executed by the apparatus for an on-site, rapid, non-destructive, and accurate identification of chemical compounds.

The present invention is illustrated by way of example and not by way of limitation in the figures of the accompanying drawings, in which the like reference numerals indicate like elements and in which:

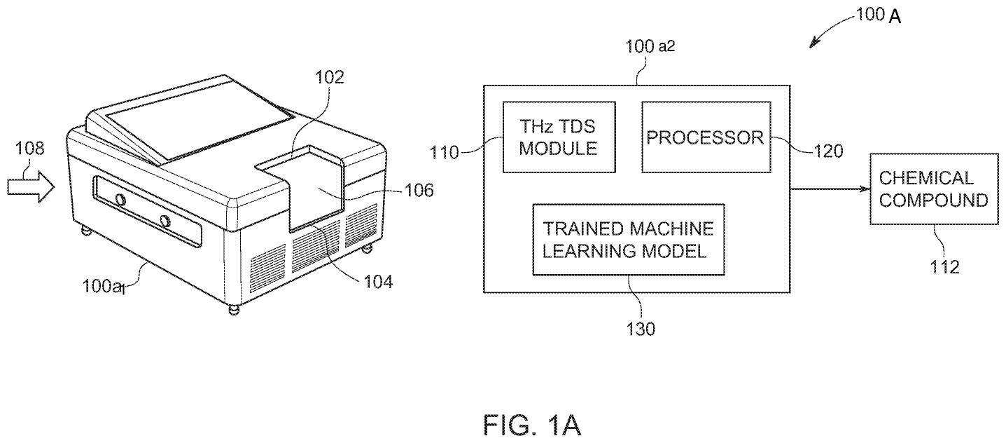

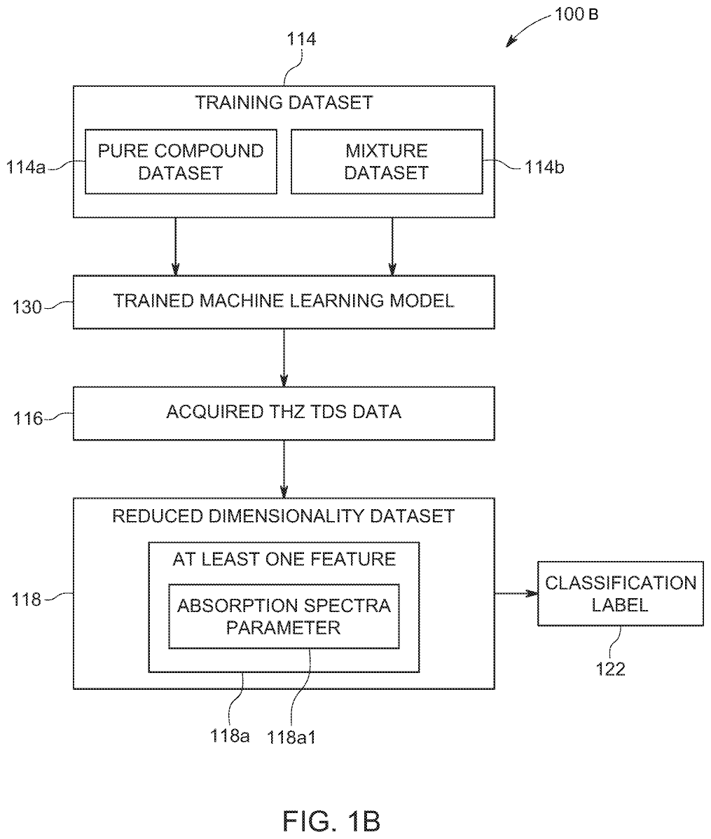

FIG. 1A illustrates a block diagram illustrating an apparatus for identifying a chemical compound from a sample, according to an example of the present disclosure;

FIG. 1B illustrates a flow diagram of training of a machine learning model of the apparatus of FIG. 1A, according to an example of the present disclosure;

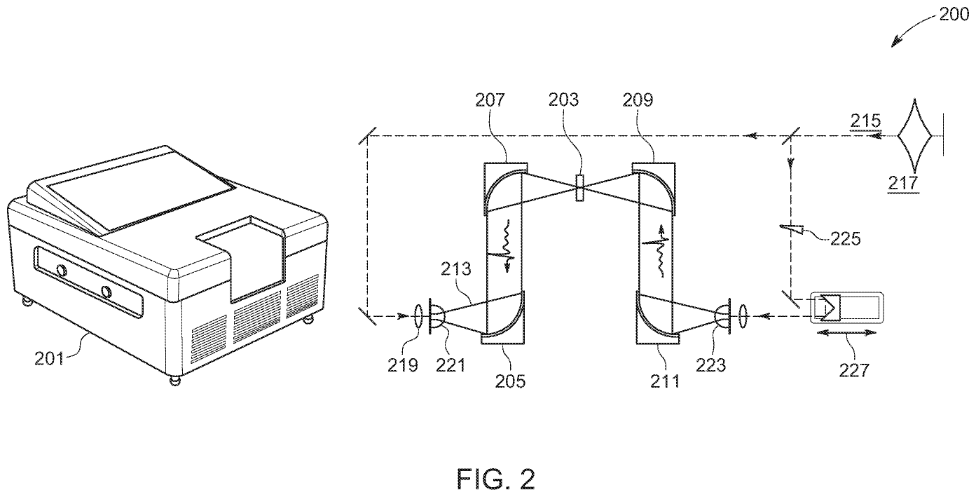

FIG. 2 illustrates a block diagram of THz-TDS broadband spectroscopy system, with schematic of the optics, according to an example of the present disclosure;

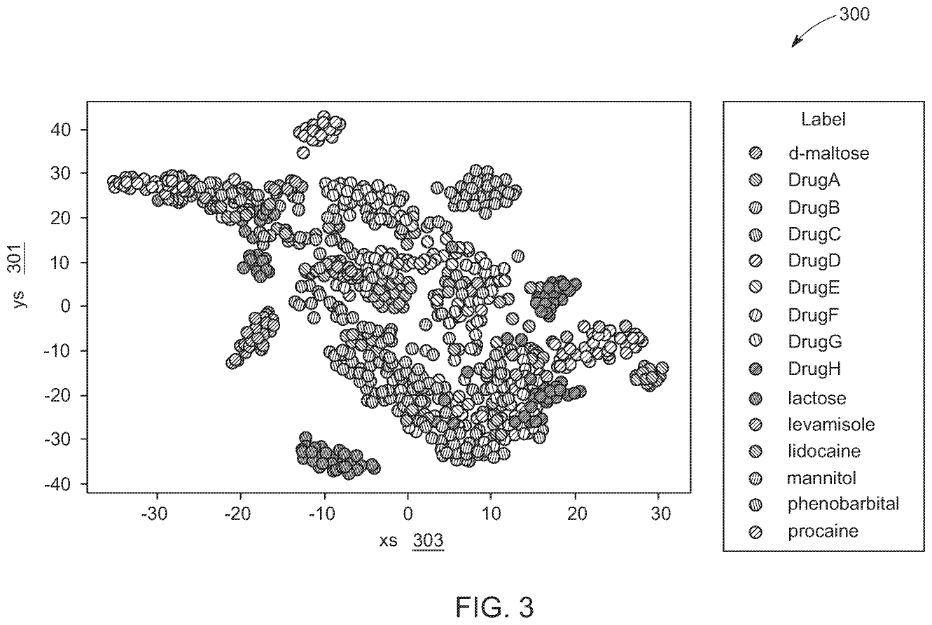

FIG. 3 illustrates a two-dimensional visualization of THz-TDS data according to an example of the present disclosure;

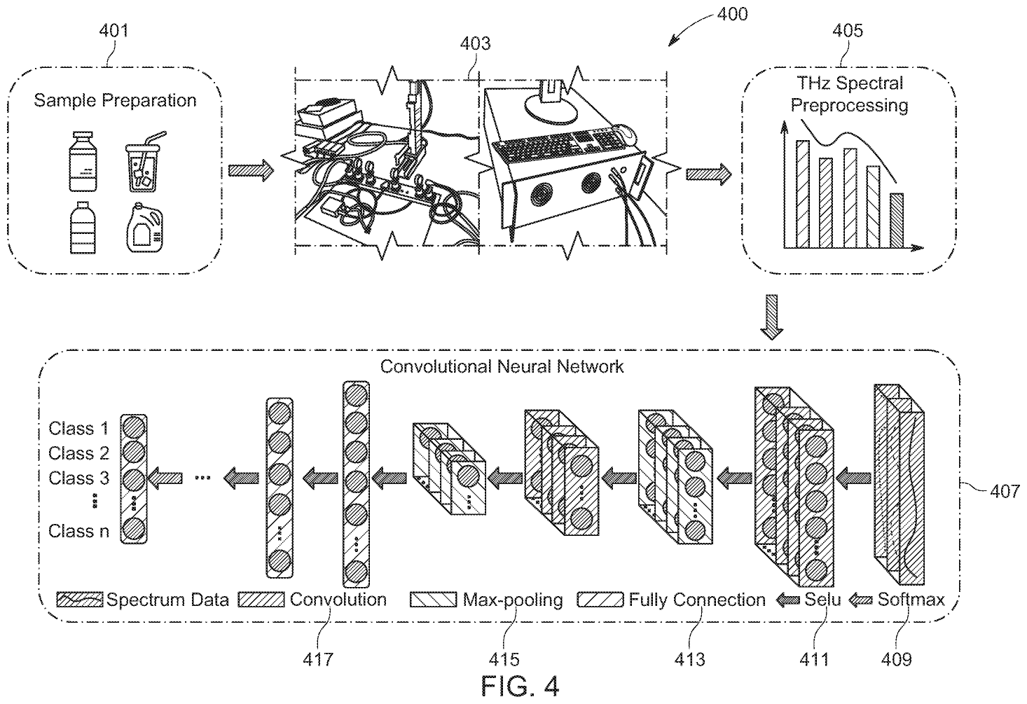

FIG. 4 is a schematic representation of a trained machine learning model performing a feature learning process and classifying the THz TDS data, according to an example of the present disclosure;

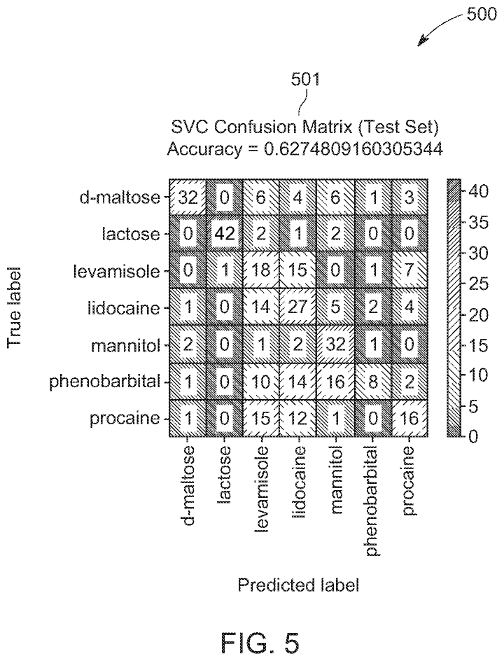

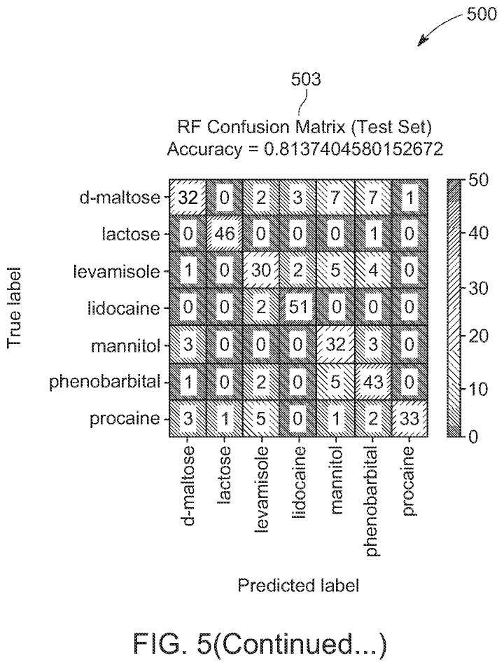

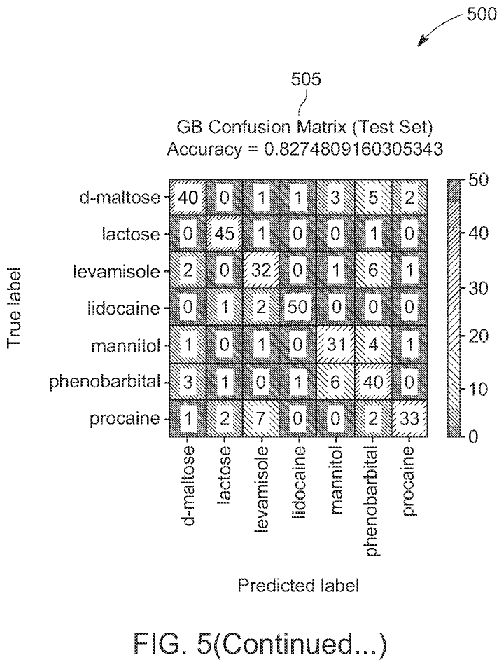

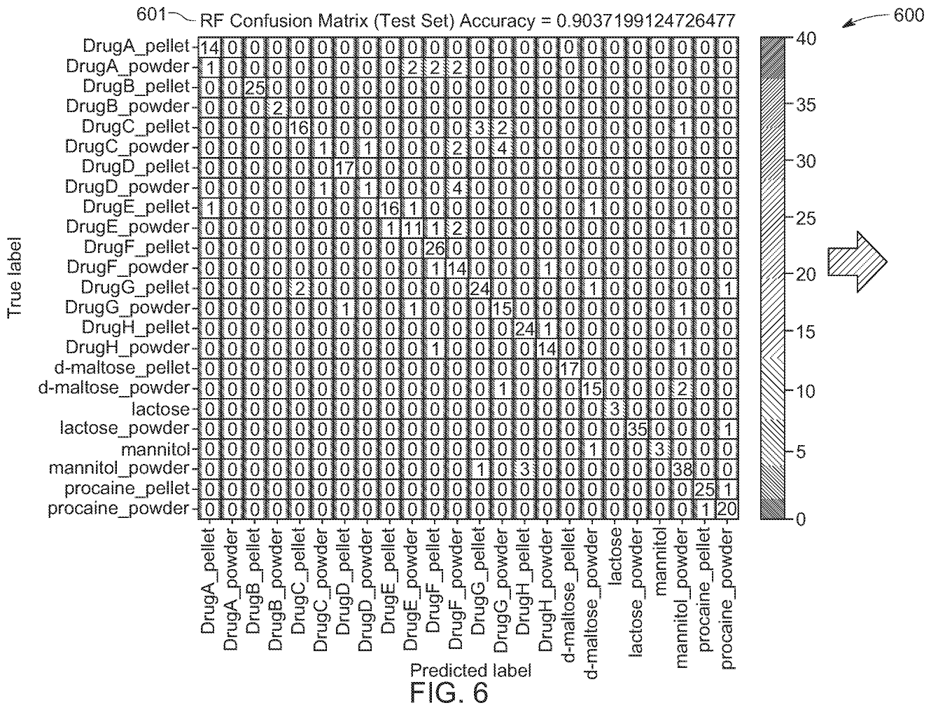

FIG. 5 illustrates a schematic representation of confusion matrices for a support vector classifier algorithm, random forest classifier algorithm, gradient boosting classifier algorithm, and neural network classifier algorithm according to an example of the present subject matter;

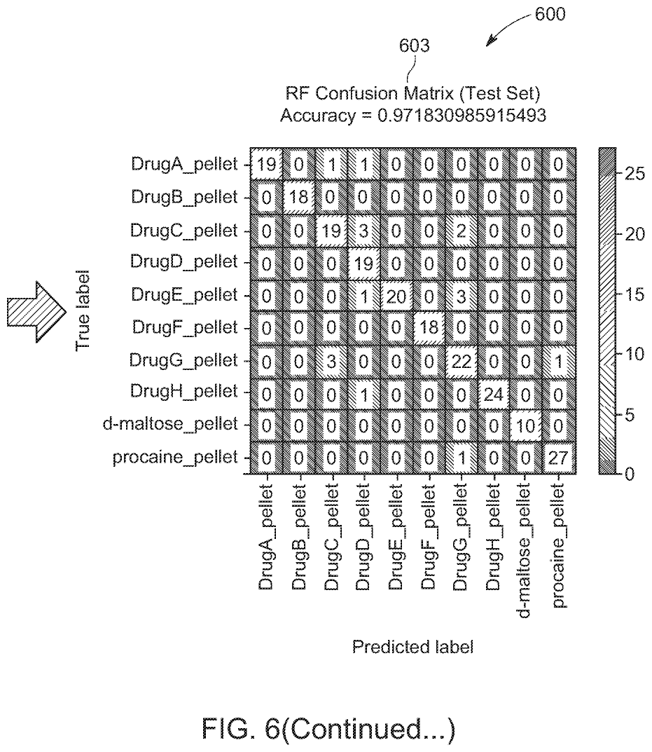

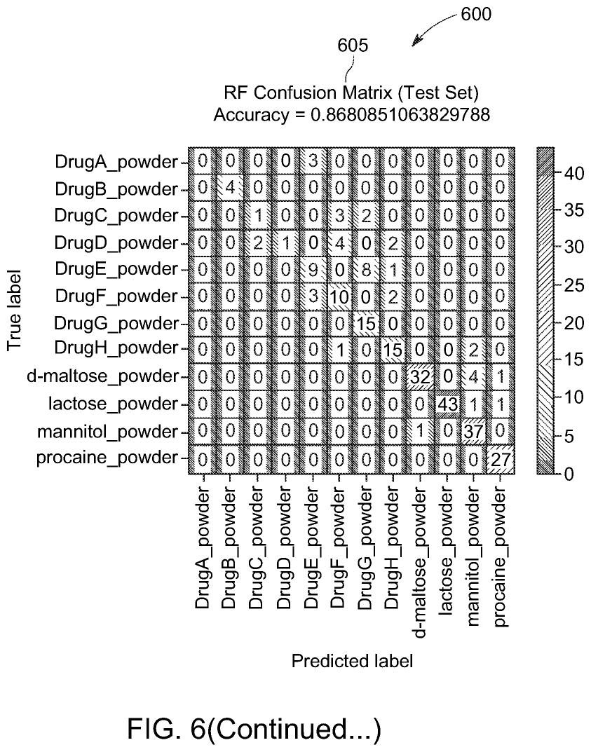

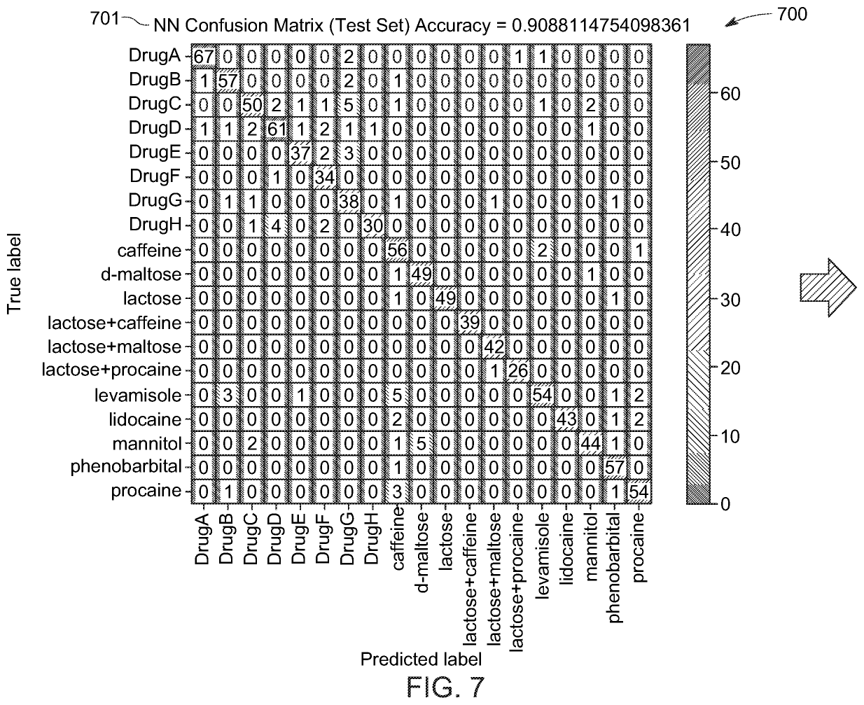

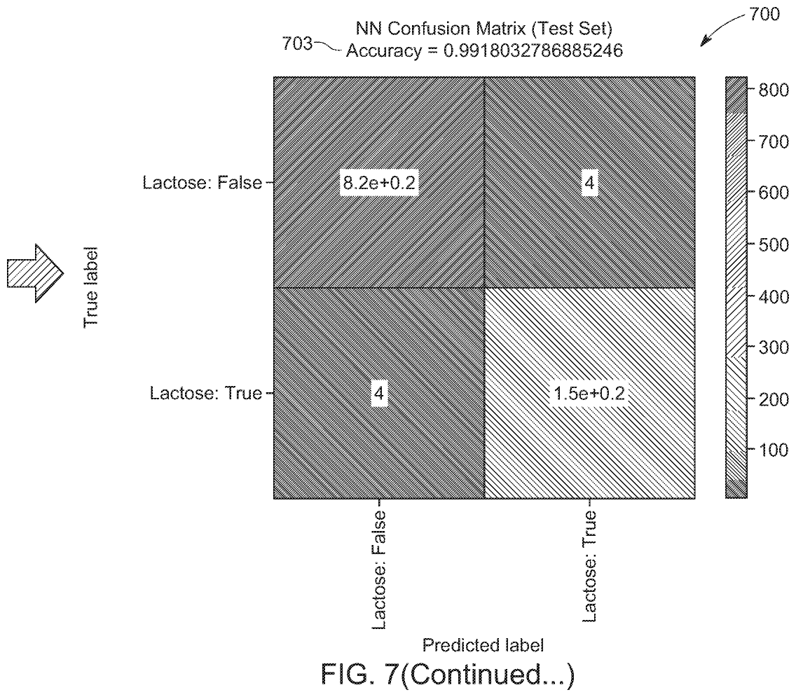

FIGS. 6 and 7 illustrate schematic representations of machine learning classification of the THz TDS data into subsets according to an example of the present subject matter; and

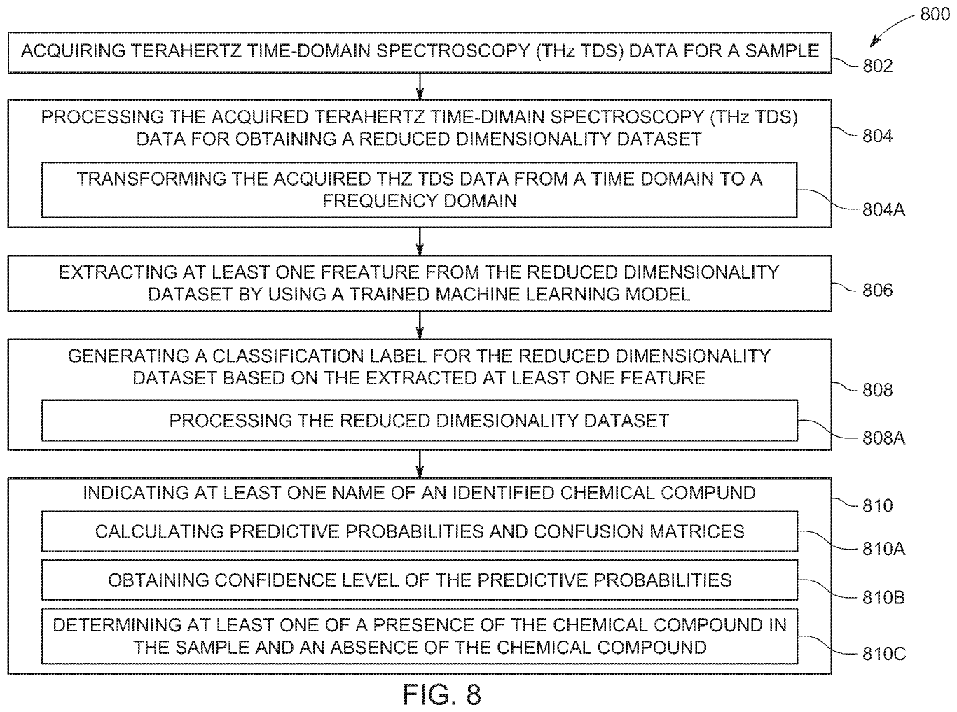

FIG. 8 illustrates a flowchart for a method for identifying a chemical compound from a sample according to an example of the present disclosure.

In the following description, for purposes of explanation, numerous specific details are set forth in order to provide a thorough understanding of the present invention. It will be apparent, however, to one skilled in the art that the present invention may be practiced without these specific details. In other instances, systems and methods are shown in block diagram form only in order to avoid obscuring the present invention.

Some aspects of the present invention will now be described more fully hereinafter with reference to the accompanying drawings, in which some, but not all, examples of the invention are shown. Indeed, various examples of the invention may be embodied in many different forms and should not be construed as limited to the examples set forth herein; rather, these examples are provided so that this invention will satisfy applicable legal requirements. Like reference numerals refer to like elements throughout. As used herein, the terms “data,” “content,” “information,” and similar terms may be used interchangeably to refer to data capable of being transmitted, received and/or stored in accordance with examples of the present invention. Further, the terms “processor”, “controller”, “processing part”, and “processing circuitry” and similar terms may be used interchangeably to refer to the processor capable of processing information in accordance with examples of the present invention. Further, the terms “electronic equipment”, “electronic devices” and “devices” are used interchangeably to refer to electronic equipment monitored by the system in accordance with examples of the present invention. Thus, use of any such terms should not be taken to limit the spirit and scope of examples of the present invention.

The examples are described herein for illustrative purposes and are subject to many variations. It is understood that various omissions and substitutions of equivalents are contemplated as circumstances may suggest or render expedient but are intended to cover the application or implementation without departing from the spirit or the scope of the present invention. Further, it is to be understood that the phraseology and terminology employed herein are for the purpose of the description and should not be regarded as limiting. Any heading utilized within this description is for convenience only and has no legal or limiting effect.

As used in this specification and claims, the terms “for example” “for instance” and “such as”, and the verbs “comprising,” “having,” “including” and their other verb forms, when used in conjunction with a listing of one or more components or other items, are each to be construed as open ended, meaning that that the listing is not to be considered as excluding other, additional components or items. Other terms are to be construed using their broadest reasonable meaning unless they are used in a context that requires a different interpretation.

One of the objectives of the present invention is to identify a chemical compound. The identification technique uses a machine learning workflow for automated compound prediction from Terahertz spectra. The Terahertz spectra are obtained by performing the terahertz time-domain spectroscopy on a sample. The Terahertz spectra are analyzed by performing spectra visualization and clustering in two dimensions. The visualization and analysis of the Terahertz spectra provides Terahertz spectra data, which is pre-processed. The pre-processing of data leads to the separation of the data in at least one data set. A prediction probability is calculated based on an existence of a chemical compound in the at least one data set. In this way, the chemical compound identification apparatus and method allows accurate determination of the chemical compound without damaging the sample.

FIG. 1A illustrates a block diagram illustrating an apparatus 100A for identifying a chemical compound 112 from a sample 106, according to an example of the present disclosure. The apparatus 100A may be implemented to check for Illicit drug detection at customs checkpoints. Further the apparatus 100A may be implemented to check for pesticide and environmental pollutants detection in drinking and wastewater. Furthermore, the apparatus 100A may be implemented for detection of harmful compounds in food products. The apparatus 100A includes physical components 100a1 and internal components 100a2.

The physical components 100a1 of the apparatus 100A comprises a cover plate 102 for focusing a terahertz beam 108 and a bottom plate 104 to enhance an absorption signal of the sample 106 to the terahertz beam 108. The sample 106 is configured to be sandwiched between the cover plate 102 and the bottom plate 104 for absorbing the terahertz beam 108.

Further, the internal components 100a2 of the apparatus 100A comprise a Terahertz time-domain spectroscopy (THz TDS) module 110, a processor 120, and a trained machine learning model 130. The THz TDS module 110 is configured to acquire terahertz time-domain spectroscopy (THz TDS) data 116 for the sample 106, where the THz TDS is performed based upon attenuated total reflection. The attenuated total reflection is a contact sampling technique that is used mostly in Fourier Transform-IR Spectroscopy to provide a quick, non-destructive sampling and also requires no sample preparation. Further, the processor 120 may be in communication with additional components, particularly a memory. The processor 120 may be any device that performs logical operations. The processor 120 may include a general processor, a central processing unit, a digital signal processor, a field programmable gate array (FPGA), a digital circuit, a controller, a microcontroller, any other type of processor or any combination thereof. The processor 120 may include one or more components operable to execute computer executable instructions or computer code embodied in the memory. The term “memory” should be interpreted broadly to encompass any electronic component capable of storing electronic information. The memory may refer to various types of processor-readable media such as random-access memory (RAM), read-only memory (ROM), non-volatile random-access memory (NVRAM), programmable read-only memory (PROM), erasable programmable read-only memory (EPROM), electrically erasable PROM (EEPROM), flash memory, magnetic or optical data storage, registers, etc.

The processor 120 is configured to implement the trained machine learning model 130. The trained machine learning model 130 is trained on a training dataset. Further, training of the trained machine learning model 130 is explained in FIG. 1B.

FIG. 1B illustrates a flow diagram 100B of training of the trained machine learning model 130 of the apparatus 100A of FIG. 1A, according to an example of the present disclosure. The trained machine learning model 130 is trained on the training dataset 114. The training dataset 114 comprises at least one pure compound (with high purity or pellet form) dataset 114a and at least one mixture (with lower purity or powder form) dataset 114b. The pure compound dataset 114a comprises data associated with the acquired THz TDS data 116 for the chemical compound 112 when the chemical compound 112 exists in a pellet form. The pure compound dataset 114a is comprised of a particular set of molecules or ions that are held together by chemical bonds which cannot be broken. In addition, these particular set of molecules or ions cannot be decomposed by ordinary chemical methods, such as the application of heat.

The mixture dataset 114b comprises data associated with the acquired THz TDS data 116 for the chemical compound 112 when the chemical compound 112 exists in a powder form. The mixture dataset 114b is comprised of two or more different substances, not chemically joined together.

The trained machine learning model 130 is configured to process the acquired THz TDS data 116. The acquired THz TDS data 116 is received from the THz TDS module 110. The acquired THz TDS data 116 is processed for obtaining a reduced dimensionality dataset 118 associated with the THz TDS data 116 by using a trained machine learning model 130. The trained machine learning model 130 is further configured to extract at least one feature 118a from the reduced dimensionality dataset 118. The at least one feature 118a comprises at least an absorption spectra parameter 118a1. Generally, absorption spectra correspond to a spectrum that is constituted by frequencies of light transmitted with dark bands when electrons absorb energy in the ground state to reach higher energy states.

The trained machine learning model 130 may perform logical operations by involving supervised machine learning, unsupervised as well as automated machine learning (will be later described in FIG. 4). The trained machine learning model 130 is configured to generate a classification label 122 for the reduced dimensionality dataset 118 based on the extracted at least one feature 118a. In an example, the reduced dimensionality dataset 118 is obtained by filtering multiple THz spectrums from the acquired THz TDS data 116 based on predetermined frequency bands. The multiple THz spectrums are filtered when the same frequency bands are processed with uniform resolution by cubic spline interpolation. In an example, the acquired THz TDS data 116 may also be filtered to remove the noise, where a smoothing filter may be applied to remove the noise without distorting the overall spectra.

The trained machine learning model 130 is further configured to indicate (e.g., displaying, showing, telling, pronouncing, expressing, etc.) a probability of identifying the chemical compound 112, based on the classification label 122 generated. The trained machine learning model 130 is further configured to determine at least one of a presence of the chemical compound 122 in the sample 106 and an absence of the chemical compound 112 in the sample 106, based on the indication of the probability of identification of the chemical compound 112.

FIG. 2 illustrates a block diagram of THz-TDS broadband spectroscopy system 200, with schematic of the optics, according to an example of the present disclosure. The THz-TDS broadband spectroscopy system 200 includes an apparatus 201, a sample 203, parabolic mirror 205, parabolic mirror 207, parabolic mirror 209, and parabolic mirror 211. In addition, the THz-TDS broadband spectroscopy system 200 includes pump light 213, beam splitter 215, femtosecond laser 217, lens 219, a detector 221, an emitter 223, a grey wedge 225, and a delay line 227. The apparatus 201 may corresponds to the apparatus 100A (100a1, 100a2) of FIG. 1A. The sample 203 corresponds to the sample 106 mentioned in FIG. 1A.

In general, parabolic mirrors provide focusing power and turning control of an input light. In an example, the pump light 213 strikes the parabolic mirror 205, 207, 209, and 211 along with the femtosecond laser 217. The beam splitter 215 splits the pump light 213, and the femtosecond laser 217 from each other. The femtosecond laser 217 emits optical pulses with femtosecond pulse durations that are transformed into a picosecond terahertz pulse. The optical pulses with femtosecond pulse durations are transformed into the picosecond terahertz pulse using the emitter 223. Further, the detector 221 is utilized to collect spectral information from the transformed picosecond terahertz pulse. Furthermore, the delay line allows changing of path difference between generation and detection of optical channels of the THz-TDS broadband spectroscopy system 200.

FIG. 3 illustrates a two-dimensional visualization 300 of the THz-TDS data 116, according to an example of the present disclosure. The two-dimensional visualization is a graphical representation of the THz-TDS data 116 along y-axis (ys) 301 and x-axis (xs) 303. As described in FIG. 1, the reduced dimensionality dataset 118 is obtained by filtering multiple THz spectrums from the acquired THz TDS data 116 based on predetermined frequency bands. In some examples, the reduced dimensionality dataset 118 corresponds to two-dimensional visual data (as shown in FIG. 3). Here the THz TDS data 116 of the sample 203 may be nonlinear in various dimensions, especially when the terahertz spectrum curves of different substances are very similar as a whole, a linear processing method is prone to large errors. Once, the reduced dimensionality dataset 118 is obtained, the processor 120 is further configured to extract the at least one feature 118a from the reduced dimensionality dataset 118 by using the trained machine learning model 130. The at least one feature 118a comprise at least the absorption spectra parameter 118a1. Generally, absorption spectra correspond to a spectrum that is constituted by frequencies of light transmitted with dark bands when electrons absorb energy in the ground state to reach higher energy states. This type of spectrum is produced when atoms absorb energy. The absorption spectra parameter 118a1 depends on thickness “d” of the sample.

Specifically, in FIG. 3, the THz spectrums together with their chemical compound label may be separated into a training set and a test set, according to a 50:50 ratio. A test set prediction accuracy may be computed as a gauge to determine the performance of the trained algorithms for predicting on unseen data. Finally, the confusion matrices may be tabulated for the various algorithms to illustrate their predictions for the various compounds.

The processing of the acquired THz TDS data 116 for obtaining the reduced dimensionality dataset 118 by the processor 120 comprises transforming the acquired THz TDS data 116 from a high dimension to a low dimension in a frequency domain, using a dimensionality reduction algorithm. The low dimension is lesser or lower than the high dimension. The low dimension may be 2 (two) in an example, while the high dimension may be any number higher than 2. In an example, the high dimension in the frequency domain of the acquired THz TDS data 116 is 100 Gigahertz (GHz). In addition, the low dimensional in the frequency domain of the acquired THz TDS data 116 is 10 Terahertz (THz). Further, the high dimension and the low dimension may not be limited to the above mentioned range. The dimensionality reduction algorithm comprises at least one of a principal component analysis algorithm or a t-distributed stochastic neighbour embedding algorithm. In general, t-distributed stochastic neighbour embedding algorithm is an unsupervised, and randomized algorithm, used only for visualization. The t-distributed stochastic neighbour embedding algorithm applies a non-linear dimensionality reduction technique where focus is on keeping the similar data points close together in lower-dimensional space.

FIG. 4 is a schematic representation 400 of the trained machine learning model 130 performing a feature learning process and classifying the THz TDS data 116, according to an example of the present subject matter. The feature learning process includes a step 401 of sample preparation. At step 401, a sample is prepared (the sample here may correspond to sample 106 of FIG. 1A). The sample 106 is configured to be sandwiched between the cover plate 102 and the bottom plate 104 of the apparatus 100A (or 201) for absorbing the terahertz beam 108. The cover plate 102 is configured to focus the terahertz beam 108 and the bottom plate 104 is configured to enhance an absorption signal of the sample 106 to the terahertz beam 108. At step 403, terahertz time-domain spectroscopy (THz TDS) data 116 is acquired for the sample 106. The terahertz time-domain spectroscopy is performed based upon attenuated total reflection. The attenuated total reflection is a contact sampling technique that is used mostly in Fourier Transform-IR Spectroscopy to provide a quick, non-destructive sampling. At step 405, the acquired THz TDS data 116 is processed with facilitation of the processor 120. The processor 120 utilizes the trained machine learning model 120 configured to process the acquired THz TDS data 116. The acquired THz TDS data 116 is received from the THz TDS module 110. The trained machine learning model 130 is further configured to extract the at least one feature 118a from the reduced dimensionality dataset 118. The at least one feature 118a comprises at least the absorption spectra parameter 118a1.

At step 407, the processed THz TDS data 116 undergoes convolution neural network to train the trained machine learning model 130. The convolution neural network includes a plurality of layers. The plurality of layers includes softmax 409, selu 411, fully-connected layer 413, max-pooling layer 415, and convolution layer 417. Generally, softmax layer turns a vector of K real values into a vector of K real values that sum to 1. The input values can be positive, negative, zero, or greater than one, but the softmax layer transforms them into values between 0 and 1, so that the input values may be interpreted as probabilities. Selu 411 corresponds to Scaled Exponential Linear Unit. In general, Selu acts as activation function for self-normalizing neural networks (SNNNs). Selu performs element-wise activation function on a given input tensor data. In general, fully connected layer multiplies each input element by weight, makes the sum, and then applies an activation function to calculate probabilities. In general, max-pooling layer reduces number of parameters and calculations in the convolution neural network. This improves the efficiency of the convolution neural network and avoids over-learning. In general, convolution layer transforms an input image in order to extract features from it. In this transformation, the image is convolved with a kernel (or filter). A kernel is a small matrix, with its height and width smaller than the image to be convolved.

The processor 120 is further configured to generate a classification label 122 for the reduced dimensionality dataset 118 based on the extracted at least one feature 118a. The classification label 122 generated for the reduced dimensionality dataset 118 comprises at least one of a true label and a false label. The true label is indicative of the presence of the chemical compound 112 in the sample 106, and the false label is indicative of the absence of the chemical compound 112 in the sample 106. The generation of the classification label 122 for the reduced dimensionality dataset 118 further comprises processing of the reduced dimensionality dataset 118 by a machine learning classification algorithm associated with the trained machine learning model 130. The machine learning classification algorithm comprising at least one of: a support vector classifier algorithm, a random forest algorithm, a gradient boosting classifier algorithm, and a neural network classifier algorithm. The trained machine learning model 130 is configured to calculate predictive probabilities and confusion matrices for the support vector classifier algorithm, the random forest algorithm, the gradient boosting classifier algorithm, and the neural network classifier algorithm for training the trained machine learning model 130. The trained machine learning model 130 is implemented with the neural network classifier algorithm having a categorical cross-entropy function to obtain confidence level of the predictive probabilities. The categorical cross-entropy function provides a measure of the confidence level of the predictive probabilities.

FIG. 5 illustrates a schematic representation 500 of confusion matrices for the support vector classifier 501, the random forest classifier 503, the gradient boosting classifier 505, and the neural network classifier algorithms according to an example of the present disclosure. Specifically in FIG. 5, the confusion matrices illustrate the predictive performance on a dataset with 7 chemical compounds. The trained machine learning model 130 is trained by calculating the predictive probabilities and confusion matrices for the support vector classifier algorithm, the random forest classifier algorithm, and the gradient boosting classifier algorithm. The actual compound is listed on y-axis, while the predicted compound is shown on x-axis. Also indicated are probabilities of the predictions. Specifically in FIG. 5, the results of doing the machine learning classification after splitting the dataset into two subsets ensure that an initial accuracy for the combined dataset was around 90%; after splitting the dataset, the pure compound dataset achieved a prediction accuracy of 97%, which is a dramatic improvement.

FIGS. 6 & FIG. 7 illustrate schematic representations 600, 700 of machine learning classification of the THz TDS data into test sets 601, 603, 605, 701, and 703 associated with the pure compound dataset and the mixture dataset, according to an example of the present subject matter. The processor 120 is further configured to generate the classification label for the reduced dimensionality dataset such as the pure compound dataset and the mixture dataset. The classification label generated for the reduced dimensionality dataset comprises at least one of a true label and a false label. The true label is indicative of the presence of the chemical compound in the sample, and the false label is indicative of the absence of the chemical compound in the sample. In an example, the pure compound dataset and the mixture dataset are labelled as “TRUE” or “FALSE” according to existence of the chemical compound mixtures in each of the pure compound and mixture datasets. In some examples, the pure compound dataset and the mixture dataset are categorized based on values related to radiation scattering and adsorption of moisture on a surface of the sample as indicated in the respective THz TDS data for each of the pure compound and the mixture dataset. Specifically, in FIG. 6 and FIG. 7, the original labels are the identities of the pure compounds or mixture of compounds, the new labels are simply “True” or “False”, where “True” indicates that a particular compound exists within the mixture and “False”, where the prediction accuracy has increased from 90% to 99% (shown in FIG. 7), indicating the effectiveness of dealing with compound mixtures in this way.

FIG. 8 illustrates a flowchart for a method 800 for identifying a chemical compound 112 from a sample 106, according to an example of the present disclosure. The method 800 may be performed by the apparatus 100A (or 201) as described in FIG. 1A, FIG. 1B, and FIG. 2 of the present disclosure. The method 800 may be implemented by a processing resource or the apparatus 100A through any suitable hardware, non-transitory machine-readable medium, or a combination thereof. In some examples, steps involved in the method 800 may be executed by the processing resource, for example the processor 120 (shown in FIG. 1). The processor 120 may be in communication with additional components. The processor 120 may include one or more components operable to execute computer executable instructions or computer code embodied in the memory. The method 800 will be described with reference to FIGS. 1A to 7.

A step 802, the method 800 comprises acquiring 802 terahertz time-domain spectroscopy (THz TDS) data of the sample 106, where the THz TDS is performed based upon attenuated total reflection. The attenuated total reflection is a contact sampling technique that is used mostly in Fourier Transform-IR Spectroscopy to provide a quick, non-destructive sampling and also requires no sample preparation.

At step 804, the method 800 comprises processing an acquired THz TDS data 116 for obtaining a reduced a reduced dimensionality dataset 118 associated with the acquired THz TDS data 116 by using the trained machine learning model 130. The trained machine learning model 130 is trained on the training dataset 114 comprising at least one pure compound (with high purity) dataset 114a and at least one mixture (with lower purity) dataset 114b. According to an example, the pure compound dataset 114a comprises data associated with the acquired THz TDS data 116 for the chemical compound 112 when the chemical compound 112 exists in a pellet form. According to an example, the mixture dataset 114b comprises data associated with the acquired THz TDS data 116 for the chemical compound 112 when the chemical compound 112 exists in a powder form.

Further, At step 804A, the trained machine learning model 130 is configured to transform 804A, using a dimensionality reduction algorithm, the acquired THz TDS data 116 from a high dimension to a low dimension in a frequency domain, and the low dimension being lesser than the high dimension, [higher to lower] [lower=less than equal to 2 in a dependent]. The dimensionality reduction algorithm comprises at least one of a principal component analysis algorithm or a t-distributed stochastic neighbour embedding (t-SNE) algorithm.

At step 806, the method 800 comprises extracting 806 at least one feature 118a from the reduced dimensionality dataset 118 by using the trained machine learning model 130. The at least one feature118a includes but may not be limited to an absorption spectra parameter 118a1.

At step 808, the method 800 further comprises generating 808 a classification label 122 for the reduced dimensionality dataset 118 based on the extraction 806 of the at least one feature 118a. In an example, the classification label 122 generated for the reduced dimensionality dataset 118 includes at least one of a true label and a false label. The true label is indicative of presence of the chemical compound 112 in the sample 106, and the false label is indicative of the absence of the chemical compound 112 in the sample 106.

According to an example, each of the true label and the false label is associated with the corresponding probability of identifying the chemical compound in the sample. For example, the true label shows probability of presence of the chemical compound in the sample, and the false label shows probability of absence of the chemical 112 compound in the sample 106.

At step 810, the method 800 further comprises indicating 810, by the trained machine learning model 130, probability of identifying the chemical compound 112 in the sample 106, based on the classification label 122 generated. The classification label 122 generated for the reduced dimensionality dataset 118 comprises at least one of the true label and the false label (as explained above). The true label is indicative of the presence of the chemical compound 122 in the sample 106, and the false label is indicative of the absence of the chemical compound 112 in the sample. At step 810A, the method 800 includes calculating 810A predictive probabilities and confusion matrices for a support vector classifier algorithm, a random forest algorithm, and a gradient boosting classifier algorithm and a neural network classifier algorithm.

According to an example, at step 810B, the method 800 further includes obtaining 810B confidence level of the predictive probabilities by implementing the neural network classifier algorithm having a categorical cross-entropy function. The categorical cross-entropy function provides a measure of the confidence level of the predictive probabilities.

At step 810C, the method 800 includes determining 810C at least one of a presence of the chemical compound 122 in the sample 106 and an absence of the chemical compound 112 in the sample 106, based on the indication 810 of the probability of identification of the chemical compound 112.

In this way, the method 800 allows accurate determination of the chemical compound without damaging the sample.

Many modifications and other examples of the inventions set forth herein will come to mind of one skilled in the art to which these inventions pertain having the benefit of the teachings presented in the foregoing descriptions and the associated drawings. Therefore, it is to be understood that the inventions are not to be limited to the specific examples disclosed and that modifications and other examples are intended to be included within the scope of the appended claims. Moreover, although the foregoing descriptions and the associated drawings describe examples in the context of certain example combinations of elements and/or functions, it should be appreciated that different combinations of elements and/or functions may be provided by alternative examples without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

### REFERENCE NUMERALS

100A—Apparatus (same as 201)
100a1—Physical components of apparatus 100A
100a2—Internal components of apparatus 100A
100B—Flow diagram of training of machine learning model
102—Copper plate
104—Bottom plate
106—Sample (same as 203)
108—A terahertz beam
110—Terahertz time-domain spectroscopy (THz TDS) module
112—Chemical compound
114—Training Dataset
114a—Pure compound dataset
114b—Mixture dataset
116—Acquired THz TDS Data
118—Reduced dimensionality dataset
118a—At least one feature
118a1—Absorption spectra parameter
120—Processor
122—Classification label
130—Trained machine learning model
200—THz-TDS broadband spectroscopy system
201—Apparatus (same as 100A)
203—Sample (same as 106)
205—Parabolic mirror (1)
207—Parabolic mirror (2)
209—Parabolic mirror (3)
211—Parabolic mirror (4)
213—Pump light
215—Beam splitter
217—Femtosecond laser
219—Lens
221—Detector
223—Emitter
225—Grey wedge
227—Delay line
300—Two-dimensional visualization of THz-TDS data
301—Y-axis (ys)
303—X-axis (xs)
400—Schematic representation of FIG. 4
401—A step of sample preparation
403-407—Step of THz TDS data processed
409—Softmax
411—Selu
413—Fully-connected layer
415—Max-pooling layer
417—Convolution layer
500—Schematic representations of FIG. 5
501—Confusion matrices for the support vector classifier
503—Confusion matrices for the random forest classifier
505—Confusion matrices for the gradient boosting classifier
600—Schematic representations of FIG. 6
601-605—THz TDS data test sets.
700—Schematic representations of FIG. 7
701-703—THz TDS data test sets
800—The method
802-810—Different steps of the method 800.

1. A method (800) for identifying a chemical compound (112) from a sample (106), the method (800) comprising:
acquiring (802) terahertz time-domain spectroscopy (THz TDS) data of the sample (106);
processing (804) an acquired terahertz time-domain spectroscopy (THz TDS) data (116) for obtaining a reduced dimensionality dataset (118) associated with the terahertz time-domain spectroscopy (THz TDS) data (116) by using a trained machine learning model (130), the trained machine learning model (130) being trained on a training dataset (114) comprising at least one pure compound dataset (114a) and at least one mixture dataset (114b);
extracting (806) at least one feature (118a) from the reduced dimensionality dataset (118) by using the trained machine learning model (130), the at least one feature (118a) comprising at least an absorption spectra parameter (118a1);
generating (808), by the trained machine learning model (130), a classification label (122) for the reduced dimensionality dataset (118) based on extraction (806) of the at least one feature (118a); and
indicating (810) by the trained machine learning model (130), a probability of identifying the chemical compound (112) in the sample (106), based on the classification label (122) generated.

2. The method (800) of claim 1, further comprising:
determining (810C) at least one of a presence of the chemical compound (112) in the sample (106) and an absence of the chemical compound (122) in the sample (106), based on the indication (810) of the probability of identifying the chemical compound (112) in the sample (106).

3. The method (800) of claim 1, wherein the absorption spectra parameter (118a1) depends on thickness “d” of the sample (106).

4. The method (800) of claim 1, wherein the processing (804) of the acquired THz TDS data (116) for obtaining the reduced dimensionality dataset (118) by using the trained machine learning model (130) further comprises:
transforming (804A), using a dimensionality reduction algorithm, the acquired THz TDS data (116) from a high dimension to a low dimension in a frequency domain, the low dimension being lower than the high dimension, wherein the dimensionality reduction algorithm comprises at least one of a principal component analysis algorithm or a t-distributed stochastic neighbour embedding algorithm.

5. The method (800) of claim 1, wherein the generating (808), by the trained machine learning model (130), the classification label (122) for the reduced dimensionality dataset (118) further comprises:
processing (808A) the reduced dimensionality dataset (118) by a machine learning classification algorithm associated with the trained machine learning, the machine learning classification algorithm comprising at least one of: a support vector classifier algorithm, a random forest algorithm, a gradient boosting classifier algorithm, and a neural network classifier algorithm.

6. The method (800) of claim 1, wherein the pure compound dataset (114a) comprises data associated with the acquired THz TDS data (116) for the chemical compound (112) when the chemical compound (112) exists in a pellet form.

7. The method (800) of claim 1, wherein the mixture dataset (114b) comprises data associated with the acquired THz TDS data (116) for the chemical compound (112) when the chemical compound (112) exists in a powder form.

8. The method (800) of claim 1, wherein the classification label (122) generated for the reduced dimensionality dataset (118) comprises at least one of a true label and a false label, wherein the true label is indicative of the presence of the chemical compound (122) in the sample (106), and the false label is indicative of the absence of the chemical compound (112) in the sample (106).

9. The method (800) of claim 1, further comprising:
calculating (810A) predictive probabilities and confusion matrices for a support vector classifier algorithm, a random forest algorithm, a gradient boosting classifier algorithm, and a neural network classifier algorithm for training the trained machine learning model (130).

10. The method (800) of claim 1, further comprising:
obtaining (810B) confidence level of the predictive probabilities by implementing the neural network classifier algorithm having a categorical cross-entropy function, wherein the categorical cross-entropy function provides a measure of the confidence level of the predictive probabilities.

11. The method (800) of claim 8, wherein the true label and the false label is associated with corresponding probability of identifying the chemical compound (112) in the sample (106).

12. The method (800) of claim 1, further comprising:
transforming (804A) the acquired THz TDS data (116) from a time domain to a frequency domain; and
the processing (804) the acquired THz TDS data (116) transformed, wherein the processing (804) of the acquired THz TDS data (116) that is transformed is performed for obtaining the reduced dimensionality dataset (118).

13. An apparatus (100A) for identifying a chemical compound (112) from a sample (106), the apparatus (100A) comprises:
a cover plate (102) for focusing a terahertz beam (108);
a bottom plate (104) for enhancing an absorption signal of the sample (106) to the terahertz beam (108),
wherein the sample (106) is configured to be sandwiched between the cover plate (102) and the bottom plate (104) for absorbing the terahertz beam (108).
a terahertz time-domain spectroscopy module (110) for acquiring terahertz time-domain spectroscopy (THz TDS) data for the sample (106); and
a processor (120) configured to implement a trained machine learning model (130), the trained machine learning model (130) being trained on a training dataset (114) comprising at least a pure compound dataset (114a) and a mixture dataset (114b), the trained machine learning model (130) configured to:
process (804) an acquired THz TDS data (116) for obtaining a reduced dimensionality dataset (118) associated with the acquired THz TDS data (116);
extract (806) at least one feature (118a) from the reduced dimensionality dataset (118), wherein the at least one feature (118a) comprises at least an absorption spectra parameter (118a1);
generate (808) a classification label (122) for the reduced dimensionality dataset (118) based on the extraction (806) of the at least one feature (118a); and
indicate (810) a probability of identifying the chemical compound (112) in the sample (106), based on the classification label (122) generated.

14. The apparatus (100A) of claim 13, wherein the trained machine learning model (130) is configured to:
determine (810C) at least one of a presence of the chemical compound (112) in the sample (106) and an absence of the chemical compound (112) in the sample (106), based on the indication (810) of the probability of identifying the chemical compound (112) in the sample (106).

15. The apparatus (100A) of claim 13, wherein the absorption spectra parameter (118a1) depends on thickness “d” of the sample (106).

16. The apparatus (100A) of claim 13, wherein the pure compound dataset (114a) comprises data associated with the acquired THz TDS data (116) for the chemical compound (112) when the chemical compound (112) exists in a pellet form.

17. The apparatus (100A) of claim 13, wherein the mixture dataset (114b) comprises data associated with the acquired THz TDS data (116) for the chemical compound (112) when the chemical compound (112) exists in a powder form.

18. The apparatus (100A) of claim 13, wherein the classification label (122) generated for the reduced dimensionality dataset (118) comprises at least one of a true label and a false label, wherein the true label is indicative of the presence of the chemical compound (112) in the sample (106), and the false label is indicative of the absence of the chemical compound (112) in the sample (106).
